(19) United States
(12) Patent Application Publication
(10) Pub. No.: US 2020/0016066 A1
(43) Pub. Date: Jan. 16, 2020

(54) SACCHARIDE-BASED ORAL MUCOADHESIVE DELIVERY SYSTEM FOR NEUROTROPHIC AND NEUROPROTECTIVE COMPOSITIONS

(71) Applicant: Food Technology and Design, LLC, DBA FoodPharma, Santa Fe Springs, AZ (US)

(72) Inventor: Timothy M. Marshall, Tucson, AZ (US)

(21) Appl. No.: 16/128,349

(22) Filed: Sep. 11, 2018

Related U.S. Application Data
(60) Provisional application No. 62/696,955, filed on Jul. 12, 2018.

(51) Int. Cl. A61K 9/00; A61K 47/26; A61K 47/44; A61K 47/12; A61K 47/24; A61K 33/06; A61K 33/00; A61K 33/30; A61K 33/04; A61K 45/06; A61K 31/194; A61K 31/375

(52) U.S. Cl. CPC A61K 9/006; A61K 47/26; A61K 47/44; A61K 47/12; A61K 47/24; A61K 31/375; A61K 33/00; A61K 33/30; A61K 33/04; A61K 45/06; A61K 31/194; A61K 33/06

(57) ABSTRACT

The oral cavity is an ideal-site for small molecule, nutrient/nutraceutical delivery to the central nervous system (CNS) and systemic circulation due to the highly-vascularized, oral mucosa, near-neutral pH conditions, close proximity to the brain, and avoidance of gastric degradation and the first-pass effect. The rich-blood supply of the oral mucosa and “sticky”, mucoadhesive properties of this novel, saccharide-based, delivery system allows for the efficient absorption of small molecules across several highly-vascularized surfaces including the gingival, sublingual, soft-palatal, and buccal mucosa. Herein, I introduce a novel, saccharide-based, food-form, palatable, easy-to-administer, oral delivery system that attaches to these four, highly-absorptive surfaces for a longer contact-duration than current orally-available formulas resulting in greater, small molecule bioavailability, and a more rapid, therapeutic effect. This invention represents the next-generation in optimized, highly-efficacious, small molecule delivery systems for CNS-related conditions that moves one-step closer in approximating intravenous injection in systemic bioavailability and therapeutic onset.

### CROSS REFERENCE TO RELATED APPLICATION

This application claims priority from U.S. Provisional Patent Application No. 62/696,955, filed Jul. 12, 2018 and entitled “A NOVEL, SACCHARIDE-BASED, ORAL MUCOADHESIVE DELIVERY SYSTEM FOR FAST-ACTING, NEUROTROPHIC AND NEUROPROTECTIVE BENEFITS,” the disclosures of which are hereby incorporated entirely herein by reference.

### BACKGROUND OF THE INVENTION

### Technical Field

This invention generally relates to delivery systems, and more particularly to a saccharide-based oral mucoadhesive delivery system for administration of neurotrophic and neuroprotective compositions.

### State of the Art

Mucoadhesion is a specific phenomenon of creating bonds during intimate contact between biological surfaces covered by a mucus layer and a mucoadhesive material. The oral bioavailability and uptake of small molecule, nutrients/nutraceuticals, is often limited by the short contact-time between the formulation and the oral mucosa, and a fast washout due to saliva flow.

Given the biodynamics of molecular transport being largely a function of a molecule's size (molecular weight; mol wt), charge, and lipophilicity—it is a well-known pharmacological/nutrient principle that neutral, smaller molecules traverse both the paracellular and transcellular transport routes into the systemic circulation more readily than charged, larger molecules. This dramatic effect of molecular size/weight on bioavailability can be readily seen in the small molecule, vitamin B6 (pyridoxine) which has a molecular weight of 169 Da and a bioavailability of 75-100% from food or supplements. In contrast, and alignment with the effect of molecular weight on bioavailability, the large-molecule, vitamin B12 (cobalamin) with a molecular weight of 1355 Da possesses an extremely low bioavailability of 1-2%. These two examples provide a general, quantitative template that can be applied to a wide variety of organic molecules of similar size/weight that clearly demonstrates the strong inverse relationship of a molecule's size/weight on mucosal absorption (i.e. the ability of a small molecule to traverse the oral and GI mucosa by simple diffusion/paracellular transport) and bioavailability.

Deficiencies of existing mucosal delivery systems, therefore, include short mucosal contact time and prolonged time required for absorption of larger molecular-weight compounds.

Accordingly, what is needed is an improved mucosal delivery system, such as for neurotrophic and neuroprotective compositions, with prolonged mucosal contact time with more rapid mucosal absorption of the delivered composition than currently available systems.

### SUMMARY OF THE INVENTION

Disclosed is a novel, highly-bioavailable, fast-acting, naturally-occurring, saccharide-based, food-form, oral (gingival, buccal, sublingual, soft-palatal), mucoadhesive, nutrient/nutraceutical delivery system for CNS-related conditions and disorders. As previously described, this delivery system is designed to work synergistically with neurotrophic and neuroprotective, small molecule nutrients and nutraceuticals.

In some embodiments, the naturally-derived, mucoadhesive, saccharide-base is comprised of tapioca syrup, palm oil, citric acid, sunflower lecithin, natural flavors, and salt. In some embodiments, the natural (or synthetically-derived) saccharide-base is comprised of isomalto-oligosaccharide (IMO) syrup combined with palm oil, citric acid, sunflower lecithin, natural flavors, and salt.

In some embodiments, the mucoadhesive, saccharide-base is comprised of any one or combination of the following naturally-occurring (or chemically-identical, synthesized) saccharide complexes or isolates: tapioca syrup, isomalto-oligosaccharide (IMO) syrup, honey, yacon syrup, agave syrup, corn syrup, coconut sugar syrup, date syrup, molasses, rice syrup, sugar cane syrup, maltitol syrup, polyglycitol syrup, sugar beet syrup, liquid inulin, allulose, tagatose, erythritol, or xylitol, combined with any combination of the following: palm oil, coconut oil, citric acid, malic acid, fumaric acid, tartaric acid, soy lecithin, sunflower lecithin, organic stevia (leaf) extract, monk fruit extract, natural colors, natural flavors.

In some embodiments, the synergistic, CNS-directed, neurotrophic and/or neuroprotective preparation contains a magnesium chelate, as one of its components. In some embodiments, the magnesium chelating compound is chosen from the group consisting of succinic acid, ascorbic acid, aspartic acid, threonic acid, lysinic acid, malic acid, tauric acid, citric acid, and gluconic acid. In some embodiments, the magnesium chelating compound is a salt of orotic acid. In some embodiments, the magnesium chelate is a salt of glycine. For maximum therapeutic efficacy, the magnesium chelate should be one of the following: glycinate, threonate, or orotate.

In some embodiments, the synergistic, CNS-directed, neurotrophic and/or neuroprotective preparation comprises a zinc chelate, as one of its components. In some embodiments, the zinc chelating compound is chosen from the group consisting of succinic acid, ascorbic acid, aspartic acid, threonic acid, lysinic acid, malic acid, tauric acid, citric acid, and gluconic acid. In some embodiments, the zinc chelating compound is a salt of orotic acid. In some embodiments, the zinc chelate is a salt of glycine. In some embodiments, the zinc chelate is a salt of methionine. For maximum therapeutic efficacy, the zinc chelate should be one of the following: glycinate, monomethionine, or orotate.

In some embodiments, the synergistic, CNS-directed, neurotrophic and/or neuroprotective preparation comprises a lithium chelate, as one of its components. In some embodiments, the lithium chelating compound is chosen from the group consisting of succinic acid, ascorbic acid, aspartic acid, threonic acid, lysinic acid, malic acid, tauric acid, citric acid, and gluconic acid. In some embodiments, the lithium chelating compound is a salt of orotic acid. In some embodiments, the lithium chelating compound is a salt of aspartic acid. For maximum therapeutic efficacy, the lithium chelate should be one of the following: glycinate, aspartate, or orotate.

In some embodiments, the synergistic, CNS-directed, neurotrophic and/or neuroprotective preparation comprises a selenium chelate or complex, as one of its components. In some embodiments, the selenium chelating compound is glycinate. In some embodiments, the selenium chelating compound is aspartate. In some embodiments, the selenium chelating agent is a non-specific amino acid or complex of amino acids (e.g. amino acid chelate). In some embodiments, the selenium chelating or complexing agent is the amino acid, methionine. In some embodiments, the selenium chelating or complexing agent is the amino acid, cysteine. For maximum therapeutic efficacy, the selenium chelate or complexing agent should be one of the following: glycinate, methionine, cysteine, or high-selenium yeast.

In some embodiments, the amount of elemental magnesium per serving is between 25 and 400 milligrams.

In some embodiments, the amount of elemental zinc per serving is between 5 and 20 milligrams.

In some embodiments, the amount of elemental lithium per serving is between 0.5 and 20 milligrams.

In some embodiments, the amount of elemental selenium per serving is between 20 and 200 micrograms.

In some embodiments, the amount of nutraceutical, phytocompounds is between 0.10 and 1000 milligrams.

Disclosed is an oral mucoadhesive delivery system having a saccharide base; a natural color; and a natural flavor.

In some embodiments, the saccharide base comprises tapioca syrup, palm oil, citric acid, and sunflower lecithin. In some embodiments, the saccharide base comprises isomalto-oligosaccharide (IMO) syrup, palm oil, citric acid, and sunflower lecithin. In some embodiments, the saccharide base comprises a saccharide from the group of saccharides consisting of: tapioca syrup, isomalto-oligosaccharide (IMO) syrup, honey, yacon syrup, agave syrup, corn syrup, coconut sugar syrup, date syrup, molasses, rice syrup, sugar cane syrup, maltilol syrup, polyglycitol syrup, sugar beet syrup, liquid inulin, allulose, tagatose, erythritol and xylol.

In some embodiments, the oral mucoadhesive delivery system further comprises a neurotrophic composition. In some embodiments, the neurotrophic composition comprises a chelate of a mineral from the group of minerals comprised of: magnesium, lithium, and zinc. In some embodiments, the neurotrophic composition comprises one or more nutraceuticals or phytocompounds (e.g. from hemp oil, echinacea, cacao) such as terpenes (e.g. limonene), phenolics (e.g. cyanidin), and various cannabinoids (e.g. anandamide).

Disclosed is an oral mucoadhesive delivery system comprising a saccharide base; and a neurotrophic composition.

In some embodiments, the mucoadhesive delivery system also comprises a chelating compound. In some embodiments, the chelating compound is from the group of chelating compounds consisting of: succinic acid, ascorbic acid, aspartic acid, threonic acid, lysinic acid, malic acid, tauric acid, citric acid, and gluconic acid.

In some embodiments, the mucoadhesive delivery system comprises magnesium. In some embodiments, the mucoadhesive delivery system comprises zinc. In some embodiments, the mucoadhesive delivery system comprises lithium.

In some embodiments, the neurotrophic composition comprises selenium; and wherein the chelating compound is from the group of chelating compounds consisting of: glycinate, methionine, and cysteine.

In some embodiments, the oral mucoadhesive delivery system further comprises a small molecule nutrient from the group of small molecule nutrients consisting of: vitamin B1, Vitamin B2, vitamin B3, vitamin B5, vitamin B6, vitamin B12, folic acid, PABA, vitamin C, Vitamin D, Vitamin E, boron, copper, chromium, selenium, and molybdenum.

In some embodiments, the oral mucoadhesive delivery system is formed into a soft-chew or a gummy.

Disclosed is an oral mucoadhesive delivery system comprising a saccharide base; a natural color; a natural flavor; and a neurotrophic composition having an elemental substance from the group of elemental substances consisting of magnesium, zinc, lithium, and selenium.

In some embodiments, the amount of elemental zinc per serving is between about 5 milligrams and about 20 milligrams. In some embodiments, the amount of elemental lithium per serving is between about 0.5 milligrams and about 20 milligrams. In some embodiments, the amount of elemental selenium per serving is between about 20 micrograms and about 200 micrograms.

The foregoing and other features and advantages of the invention will be apparent to those of ordinary skill in the art from the following more particular description of the invention and the accompanying drawings.

### BRIEF DESCRIPTION OF THE DRAWINGS

A more complete understanding of the present invention may be derived by referring to the detailed description and claims when considered in connection with the Figures, wherein like reference numbers refer to similar items throughout the Figures.

FIG. 1 is a diagram illustrating trans-mucosal absorption of a composition from the oral cavity;

FIG. 2 is a diagram illustrating a mucoadhesive delivery system used in a buccal and gingival position within the oral cavity;

FIG. 3 is a diagram illustrating a mucoadhesive delivery system used in a sublingual position within the oral cavity; and

FIG. 4 is a diagram illustrating a mucoadhesive delivery system used in a soft palatal position within the oral cavity.

### DETAILED DESCRIPTION OF EMBODIMENTS OF THE INVENTION

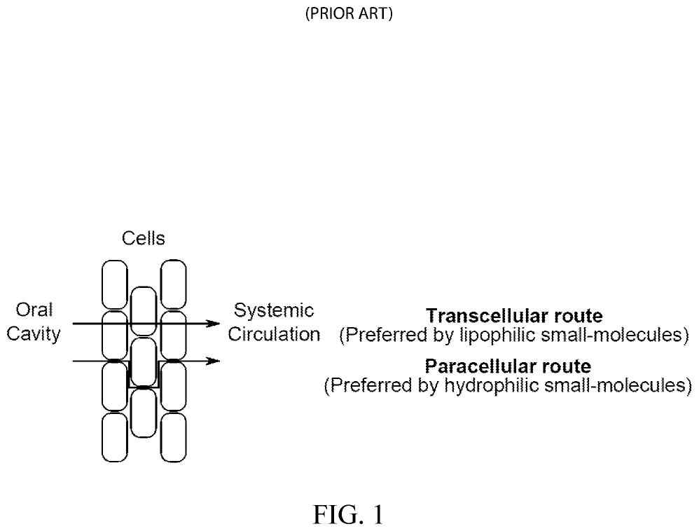

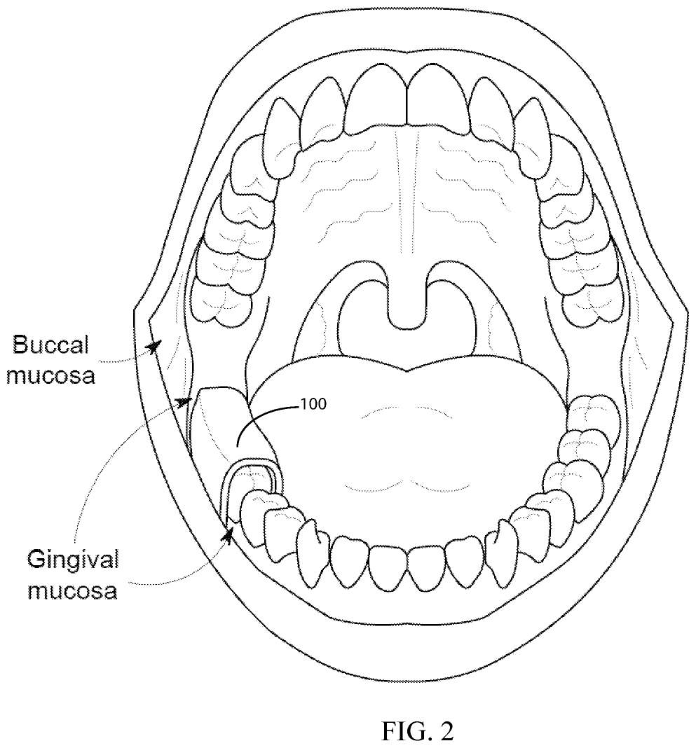

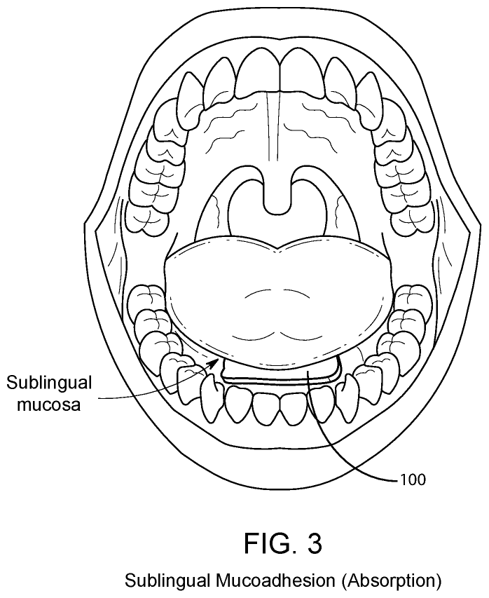

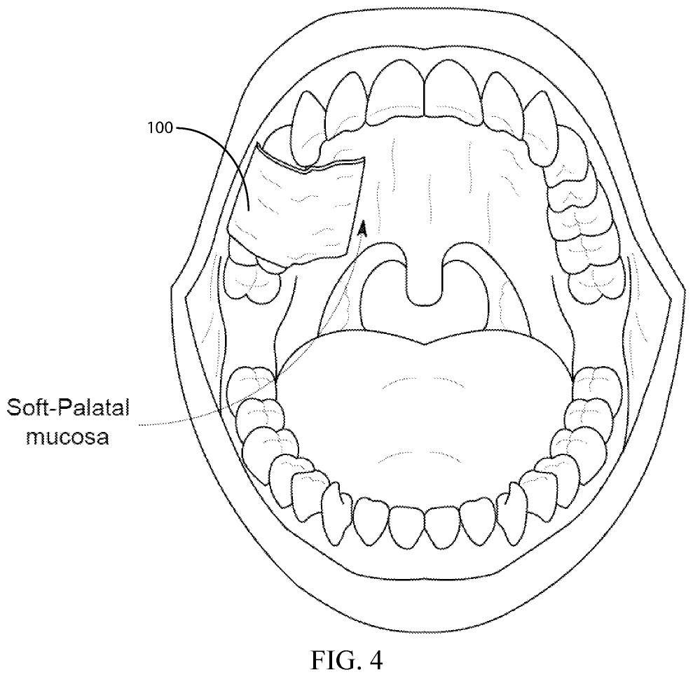

As noted herein above, embodiments of the present invention relate to a novel, fast-acting, highly-bioavailable, food-form (food-grade), saccharide-based (e.g. mono-, oligo-, polysaccharide), neurotrophic and neuroprotective, nutrient/nutraceutical delivery system 100 for central nervous system (CNS)-related conditions or disorders. Through its innate, residence (contact-exposure) increasing, mucoadhesive properties—the novel, saccharide-based delivery system 100 efficiently increases small molecule, paracellular and transcellular transport (see FIG. 1) across four, highly-vascularized, oral-mucosal surfaces (e.g. gingival, buccal, sublingual, soft-palatal) for rapid systemic delivery and therapeutic onset (see FIG. 2-4). Due to its rich supply of blood vessels, near-neutral pH, and close proximity to the brain—the oral-mucosal tissues represent an ideal site for systemic, small molecule delivery—especially for CNS-related conditions involving depression, anxiety, sensitivity to stress, attention deficit hyperactivity disorder (“ADHD”), cognitive impairment, neuroinflammation, NMDA-receptor hyperactivity, endocannabinoid deficiency, heavy-metal toxicity, chronic pain, and sleep disturbances where rapid therapeutic intervention and onset is desired.

Small molecule bioavailability and therapeutic onset are two important factors in the effectiveness of any nutritional or nutraceutical supplement with intended, CNS-directed, neurotrophic or neuroprotective therapeutic benefits (e.g. antidepressant effect, stress-reducing effect, calming effect, anti-inflammatory effect, antioxidant activity, reduction of oxidative-stress, pain relief, NMDA receptor inhibition).

For CNS-related conditions such as depression, anxiety, brain and nerve injury, headaches, chronic pain, and cognitive impairment where nutrient deficiencies and neuro-inflammation play a strong, contributing role—nutrient bioavailability and onset of therapeutic effect are of great value to the consumer. Non-toxic, technologies that improve bioavailability and speed of therapeutic onset are thus highly-desirable characteristics in any nutritional or nutraceutical product that has the ability to produce a valuable therapeutic effect.

A “sticky,” mucoadhesive, food-form, saccharide-based, “soft-chew” or “gummy” delivery system has three primary advantages over current oral technologies—(e.g. quick-dissolve tablets, non-mucoadhesive sublingual tablets, non-mucoadhesive lozenges, powder packets for liquid delivery)—designed for greater nutrient/nutraceutical bioavailability.

Advantages of the novel delivery system 100 include: 1. Increased residence time (i.e. extended contact-duration, exposure time of nutrients, directly in contact with the oral mucosa): due to the “sticky”, extended-oral-mucosal-contact-duration and multi-surface interaction (e.g. buccal, gingival, sublingual, soft-palatal) coupled to the close proximity to the brain—this novel, delivery system provides rapid, therapeutic onset for CNS-related conditions involving depression, anxiety, sensitivity to stress, attention deficit hyperactivity disorder (“ADHD”), cognitive impairment, neuroinflammation, NMDA-receptor hyperactivity, endocannabinoid deficiency, heavy-metal toxicity, chronic pain, and sleep disturbances where rapid therapeutic intervention and onset is desired.

This next-generation, oral delivery system exploits the highly-vascularized, oral mucosa, near-neutral pH, and close proximity to the brain to quickly and efficiently transport stress-reducing (i.e. anxiolytic), mood-boosting (i.e. anti-depressant), anti-inflammatory, small molecule nutrients (e.g. selenium glycinate, zinc glycinate, lithium orotate), nutraceuticals (e.g. phytocompounds: terpenes, phenolics, cannabinoids), and other similarly therapeutic, small molecule nutritional elements for maximum consumer benefit and therapeutic effect.

2. Unique biodynamics of delivery system: due to the bulky (˜5.2-5.8 g) and “sticky” nature of each “soft-chew” or “gummy”—when chewed, the mucoadhesive “soft-chew” or “gummy” spreads and extends over the upper and lower teeth, adhering to—making direct and prolonged contact with the gums (gingiva), inner sides of the cheeks (buccal), underside of the tongue (sublingual), and roof of the mouth (soft-palatal) (FIGS. 2-4).

3. Synergistic mucoadhesive, small molecule delivery system: this novel, mucoadhesive delivery system works synergistically with the following therapeutic, small molecule nutrients (mol wt<500 Da): lipophilic, neutral (uncharged), stable (poorly-ionized), mineral chelates (e.g. glycinates, mol wt<250 Da; lithium orotate, mol wt=162 Da, respectively), retinol (mol wt: 286 Da), thiamine (mol wt: 265 Da), riboflavin (mol wt: 376 Da), niacinamide (mol wt: 122 Da), pantothenic acid (mol wt: 219 Da), pyridoxine (mol wt: 169 Da), folinic acid (mol wt: 473 Da), folic acid (mol wt: 441 Da), vitamin C (mol wt: 176 Da), vitamin D (mol wt: 387 Da), and lipophilic, nutraceuticals such as terpenes (mol wt<230 Da), phenolics (mol wt<200 Da), and cannabinoids (mol wt<350 Da)—which possess greater bioavailability at the mucoadhesive-mucosa interface than larger, less-lipophilic molecules (e.g. zinc citrate, mol wt=574 Da).

For more rapid onset, and maximum therapeutic benefit and efficacy—lipophilic, uncharged, poorly-ionized, mineral chelates such as those bound to orotate or glycinate are the preferred vehicles (carriers) for efficient, mineral delivery and absorption (i.e. high-bioavailability). Small molecule, mineral chelates such as those bound to orotate (e.g. Li-orotate, mol wt: 162 Da; divalent ions, di-orotates of Ca, Mg, Zn<410 Da) or glycinate (e.g. bisglycinates of Ca, Mg, Cu, Mn, Mo, Se<250 Da) are absorbed via hydrophilic, paracellular transport and lipophilic, transcellular transport, and/or through transcellular, carrier-mediated orotate or glycine transporters.

A 1978 study comparing the bioavailability of lithium orotate to lithium carbonate demonstrated 3 times greater bioavailability for poorly-ionized (lipophilic) lithium orotate as compared to highly-ionized (hydrophilic) lithium carbonate. This study illustrates a primary distinction between the orotate vs carbonate forms of lithium, showing that the poorly-ionized, uncharged, orotate form more readily crosses lipophilic barriers (cell membranes) compared to the highly-ionized, charged, carbonate form—as evidenced by a 3-times higher level in the brain. With our present understanding of small-molecule bioavailability, this result can be reliably predicted and applied to a wide range of molecules with similar (low mol wt, neutral, uncharged, lipophilic; high-bioavailability) and dissimilar (high mol wt, ionized, charged, hydrophilic; low-bioavailability) physicochemical properties.
In further support of the effect of ionizability (i.e. ability to form charged ions) and molecular weight/size on bioavailability—a 2008 study investigating the acute uptake (ie. bioavailability) of four different forms of zinc (e.g. oxide, picolinate, gluconate, glycinate) found that zinc glycinate had the highest bioavailability. The following is the ionizability and molecular weight for each form: Zn-oxide (highly-ionized; mol wt: 81 Da), Zn-picolinate (moderately ionized; mol wt: 310 Da), Zn-gluconate (highly-ionized; mol wt: 456 Da), and Zn-glycinate (poorly-ionized; mol wt: 214 Da). Plasma zinc rankings based on area under the curve, as well as by rank results per person, were: glycinate>gluconate>picolinate=oxide. A +43.4% increase in bioavailability was seen for zinc glycinate over the second, most-bioavailable form, zinc gluconate.
In summary, lithium orotate (162 Da) and zinc glycinate (214 Da) demonstrate superior bioavailability due to their small size (low molecular weight) and both being stable, neutral chelates as compared to the unstable, highly-ionized, charged variants (e.g. lithium carbonate, zinc oxide) of both forms.

Neutral, small molecule, nutrient/nutraceutical transport occurs through both a paracellular transport mechanism across the tight junctions between cells, and directly across the lipid-bilayer of cell membranes via transcellular transport. Their low-molecular weight (i.e. small size) favors and promotes paracellular transport, while their neutral charge (coupled with small size) permits highly-efficient, transcellular (transmucosal) transport into the systemic circulation.

Stable, lipophilic, mineral chelates (e.g. glycinates, orotates) of magnesium, zinc, and lithium share similar physicochemical properties, in terms of size and lipophilicity, as that of known, highly-bioavailable, small molecules (mol wt<500 Da) such as caffeine (mol wt: 194 Da) and nicotine (mol wt: 162 Da)—with molecular weights between 162 Da (lithium orotate) to 214 Da (zinc glycinate). Nutraceuticals such as terpenes (e.g. limonene, 136 Da), phenolics (e.g. cyanidin, 287 Da), and cannabinoids (e.g. anandamide, 348 Da) also share similar physicochemical properties.

As discussed above, the disclosed invention relates to a novel, highly-bioavailable, fast-acting, saccharide-based, multi-surface, oral (gingival, buccal, sublingual, soft-palatal), mucoadhesive delivery system for CNS-related conditions and disorders.

An example of a synergistic, neurotrophic and/or neuroprotective, small molecule, nutrient/nutraceutical preparation functioning in-tandem with the novel, mucoadhesive delivery system described herein might consist of (but is not limited to): magnesium glycinate, lithium orotate, zinc glycinate, thiamine, riboflavin, niacin, pantothenic acid, pyridoxine, folinic acid, cannabinoids (e.g. from hemp oil extract, echinacea, cacao), and various phytocompounds from plants (e.g. anthocyanins, terpenes, phenolics). Such a preparation would exert a number of neurotrophic and/or neuroprotective therapeutic benefits (e.g. antidepressant effect, stress-reducing effect, calming effect, anti-inflammatory effect, antioxidant activity, reduction of oxidative-stress, pain relief, NMDA receptor inhibition) in humans and animals.

The neurotrophic and/or neuroprotective preparation briefly described above functioning in biological synergy with the novel delivery system described herein—with its rapid therapeutic onset and effect—would be of great value and benefit to those with CNS-related conditions involving depression, anxiety, sensitivity to stress, attention deficit hyperactivity disorder (“ADHD”), cognitive impairment, neuroinflammation, NMDA-receptor hyperactivity, endocannabinoid deficiency, heavy-metal toxicity, chronic pain, and sleep disturbances where rapid therapeutic intervention is desired.

The mucoadhesive, saccharide complex or isolate can be either naturally-derived (e.g. tapioca syrup, rice syrup, isomalto-oligosaccharide (IMO) syrup) and/or chemically-identical, synthetically-derived.

Magnesium, lithium, zinc, and cannabinoids (e.g anandamide) exert a portion of their neuroprotective, antioxidant, anti-inflammatory effects through inhibition of the N-methyl-D-aspartate receptor (“NMDA”), with which magnesium (and/or lithium, zinc) interact as a receptor ligand, on post-synaptic cortical neurons of the central nervous system. With their innate antioxidant activity, and CB1/CB2 receptor binding affinity, cannabinoids are believed to exert their NMDA inhibitory effect through a different mechanism. NMDA receptors are ubiquitous throughout the brain and play a role in regulation of the excitatory state of post-synaptic neurons. NMDA receptors act as a cationic membrane “pore,” primarily for calcium ions although other cations such as sodium, zinc, and protons may pass into the cell. In conditions wherein the post-synaptic neuron is polarized and glutamate is absent from the synapse, a local negative membrane charge permits the pore to be blocked with a magnesium ion. Under conditions wherein 1) glutamate is present within the synapse at a sufficient concentration; and 2) the post-synaptic neuron is partially depolarized creating a neutral or relative positive membrane charge, the magnesium ion is displaced, the pore opens, and calcium ions are allowed to pass freely through the NMDA receptor into the cell. Once intracellular, calcium exerts a myriad of secondary effects, largely through its role as a secondary messenger and enzyme cofactor. Increased intracellular calcium leads to increased cellular enzyme activity of proteases, nucleases, and phospholipases, breaking down structural components and functional machinery of the cell and often leading to cell death.

Keeping the baseline state of the NMDA receptor pore in a closed configuration, therefore, is important for the proper function and survival of a post-synaptic neuron. There are at least two potential sites of action to keep the receptor pore closed to influx of calcium and other cations into the neuron: 1) adequate-to-high synaptic magnesium concentrations; and 2) tyrosine-mediated phosphorylation of the NR2B receptor subunit.

Given a polarized or neutral post-synaptic cell membrane, in combination with adequate extracellular magnesium concentrations, magnesium binds to the receptor pore and blocks influx of calcium.

Several physiologic mechanisms resist a partially depolarized state in the post-synaptic cell membrane, keeping the pore closed to the influx of calcium an enhancing appropriate NMDA receptor function. One of these potentiating mechanisms is tyrosine-mediated phosphorylation of the NMDA receptor subunits, tending to “close” the receptor pore by causing an amphoteric shift in one or more protein subunits. Tyrosine phosphatase-mediated NR2B subunit phosphorylation potentiated by the lithium cation has been shown to cause depression of NMDA receptor currents.

The present invention, in addition to exceptional bioavailability with a broad-spectrum of neurotrophic effects (e.g. increased neural growth factors (e.g. BDNF), stem cell proliferation, Nrf2 activation, enhanced antioxidant defenses (e.g. GPx), essential nutrient repletion) seeks to militate against activation of a final common pathway for neuronal cell injury and death—elevated intracellular calcium levels—by reducing oxidative stress and neural inflammation, and impeding permeability of the NMDA receptor to calcium.

Magnesium concentrations in a neuronal synapse are necessary to saturate the post-synaptic population of NMDA receptors, therein keeping the receptor pores closed to the influx of extracellular calcium ions when the post-synaptic neuron is in a partially polarized state. Hypomagnesaemia is associated with a plethora of symptomatic neurological abnormalities, such as depression, anxiety, sleep disturbances, hyperreflexia, tremor, confusion, hallucinations, convulsions, hyperacusis, nystagmus, tetany, delirium tremens, and extrapyramidal disorders.

Trans-mucosal absorption of elemental magnesium is greatly increased by combining elemental magnesium with a stable, organic chelate. In some embodiments, the magnesium chelating compound is a salt of glycine. In some embodiments, the magnesium chelating compound is a salt of orotic acid. In some embodiments, the magnesium chelating compound is a salt of succinic acid, aspartic acid, ascorbic acid, threonic acid, gluconic acid, lysinic acid, malic acid, tauric acid, or citric acid. It is anticipated that as experimentation and research in the art of enhanced trans-mucosal magnesium absorption progresses, other chelating compounds may be used as a chelating compound, in some embodiments.

The total concentration of elemental magnesium per dose of the neuroprotective preparation is calculated to provide adequate intracerebral levels of magnesium without being so high as to increase the risk of toxicity from hypermagnesaemia. Accordingly, the amount of elemental magnesium, in some embodiments, is between approximately 25 milligrams and 400 milligrams.

The synergistic nutrients and/or nutraceuticals used with this novel, mucoadhesive delivery system can by comprised, but not limited to, any one or combination of the following: vitamins, minerals, nutrients, or nutraceuticals used for their therapeutic, neurotrophic and/or neuroprotective benefits, as previously described above. In some embodiments, the proprietary neurosupportive blend comprises (per serving): magnesium glycinate, 25-400 mg; lithium orotate, 0.5-20 mg; boron glycinate, 0.5-10 mg; copper glycinate, 0.25-2 mg; zinc glycinate, 5-20 mg; manganese glycinate, 0.5-5 mg; selenium glycinate, 25-200 mcg; molybdenum glycinate, 25-200 mcg; and chromium polynicotinate, 25-400 mcg; kelp, 10-100 mg; vitamin B1, 1-25 mg; vitamin B2, 1-25 mg; vitamin B6, 1-25 mg; Vitamin B3, 5-100 mg; vitamin B5, 5-200 mg; Vitamin B12 as methylcobalamin, hydroxocobalamin, adenosylcobalamin; folate as folic acid, methyl-folate, or folinic acid, 25-400 mcg; 25-1,000 mcg; non-GMO vitamin C as ascorbic acid, ascorbyl palmitate, or ascorbates, 50-1,000 mg; vitamin D3, 500-5,000 IU; and nutraceuticals such as terpenes (e.g. limonene), phenolics (e.g. cyanidin), and cannabinoids (e.g. anandamide).

The nutrients and/or nutraceuticals used in some embodiments are comprised of some of the most bioavailable nutrients (e.g. Albion® minerals) along with the novel, mucoadhesive delivery system previously described facilitating exceptional, oral bioavailability through the gingival, buccal, sublingual, and soft-palatal mucosa into the systemic circulation.

A novel, synergistic, fast-acting, highly-bioavailable, saccharide-based, mucoadhesive, neurotrophic and/or neuroprotective delivery system for CNS-related conditions or disorders, and methods of use has been described. This invention represents the next-generation in optimized, highly-efficacious, small molecule delivery systems for CNS-related conditions that moves one-step closer in approximating intravenous injection in systemic bioavailability and therapeutic onset.

1. An oral mucoadhesive delivery system comprising:
a saccharide base;
a natural color; and
a natural flavor.

2. The oral mucoadhesive delivery system of claim 1, wherein the saccharide base comprises:
tapioca syrup, palm oil, citric acid, and sunflower lecithin.

3. The oral mucoadhesive delivery system of claim 1, wherein the saccharide base comprises:
isomalto-oligosaccharide (IMO) syrup, palm oil, citric acid, and
sunflower lecithin.

4. The oral mucoadhesive delivery system of claim 1, wherein the saccharide base comprises a saccharide from the group of saccharides consisting of: tapioca syrup, isomalto-oligosaccharide (IMO) syrup, honey, yacon syrup, agave syrup, corn syrup, coconut sugar syrup, date syrup, molasses, rice syrup, sugar cane syrup, maltilol syrup, polyglycitol syrup, sugar beet syrup, liquid inulin, allulose, tagatose, erythritol and xylol.

5. The oral mucoadhesive delivery system of claim 4, further comprising a neurotrophic composition.

6. The oral mucoadhesive delivery system of claim 5, wherein the neurotrophic composition comprises a chelate of a mineral from the group of minerals comprised of: magnesium, lithium, and zinc.

7. The oral mucoadhesive delivery system of claim 5, wherein the neurotrophic composition comprises a cannabinoid, an active phytocompound, a phenolic, and a terpene.

8. An oral mucoadhesive delivery system comprising:
a saccharide base; and
a neurotrophic composition.

9. The oral mucoadhesive delivery system of claim 8, comprising a chelating compound.

10. The oral mucoadhesive delivery system of claim 9, wherein the chelating compound is from the group of chelating compounds consisting of: succinic acid, ascorbic acid, aspartic acid, threonic acid, lysinic acid, malic acid, tauric acid, citric acid, and gluconic acid.

11. The oral mucoadhesive delivery system of claim 10, comprising magnesium.

12. The oral mucoadhesive delivery system of claim 10, comprising zinc.

13. The oral mucoadhesive delivery system of claim 10, comprising lithium.

14. The oral mucoadhesive delivery system of claim 9, wherein the neurotrophic composition comprises selenium; and wherein the chelating compound is from the group of chelating compounds consisting of: glycinate, methionine, and cysteine.

15. The oral mucoadhesive delivery system of claim 8, further comprising a small molecule nutrient from the group of small molecule nutrients consisting of: vitamin B1, Vitamin B2, vitamin B3, vitamin B5, vitamin B6, folic acid, PABA, Vitamin C, vitamin D, vitamin E, boron, copper, chromium, selenium, and molybdenum.

16. The oral mucoadhesive delivery system of claim 8 formed into a soft-chew or a gummy.

17. An oral mucoadhesive delivery system comprising:
a saccharide base;
a natural color;
a natural flavor; and
a neurotrophic composition having an elemental substance from the group of elemental substances consisting of magnesium, zinc, lithium, and selenium.

18. The oral mucoadhesive delivery system of claim 17, wherein the amount of elemental zinc per serving is between about 5 milligrams and about 20 milligrams.

19. The oral mucoadhesive delivery system of claim 17, wherein the amount of elemental lithium per serving is between about 0.5 milligrams and about 20 milligrams.

20. The oral mucoadhesive delivery system of claim 17, wherein the amount of elemental selenium per serving is between about 20 micrograms and about 200 micrograms.
